(19) United States
(12) Patent Application Publication
(10) Pub. No.: US 2019/0329004 A1
(43) Pub. Date: Oct. 31, 2019

(54) FLEXIBLE CATHETER USING WIRES

(71) Applicant: GSMT CO., LTD., Gwangju (KR)

(72) Inventors: Jung Won SHIN, Gwangju (KR); Cheong Cheon LEE, Gwangju (KR)

(73) Assignee: GSMT CO., LTD., Gwangju (KR)

(21) Appl. No.: 16/462,220

(22) PCT Filed: Jul. 25, 2017
(86) PCT No.: PCT/KR2017/007999

§ 371 Date: May 18, 2019

(30) Foreign Application Priority Data
Nov. 18, 2016 (KR) 10-2016-0154130

(51) Int. Cl. A61M 25/01; A61M 25/00

(52) U.S. Cl. CPC A61M 25/0147; A61M 2025/015; A61M 25/008; A61M 25/0054

(57) ABSTRACT

The present invention relates to a flexible catheter comprising: an insertion unit comprising multiple bending parts disposed in a lengthwise direction; an operation unit connected to the insertion unit; direction-controlling wires which have an end connected to the operation unit and the other end fixed to the insertion unit and are configured so as to bend the bending parts according to the length pulled by the operation unit; and limiters provided on the direction-controlling wires and configured such that the respective multiple bending parts are sequentially bent as the direction-controlling wires are pulled. The flexible catheter using wires, according to the present invention, allows for smooth steering of 180 degrees or more, is capable of sequentially bending the bending parts and thus steering to an angle optimized for a particular tissue, and therefore has the effect of efficiently and accurately carrying out a surgical operation.

### TECHNICAL FIELD

The present invention relates to a flexible catheter using a wire, and more particularly to a flexible catheter which uses a wire and realizes bending in a plurality of portions thereof.

### BACKGROUND ART

A catheter is a tube-shaped instrument that is mainly inserted into a body cavity or a body organ upon a surgical operation, and is usually referred to as a probe. The catheter is formed of various materials to have various sizes and shapes according to the use purpose thereof. Such a catheter may be classified, according to the hardness thereof, into a solid catheter that is hard and an elastic flexible catheter.

The catheter may be used to insert an optical fiber through a working channel therein to photograph the inside of a body organ or to insert a certain treatment unit in order to perform appropriate treatment. In this case, the catheter tip is steered to approach a lesion site at an appropriate angle. For example, a wire is used for steering.

Korean Patent Registration No. 1,331,523 filed by the present applicant in connection with the aforementioned flexible catheter is published.

In relation to a small-diameter catheter that is inserted into a narrow body cavity such as an ankle joint, in particular, conventional flexible catheters have difficulty in achieving a steering angle of 180 degrees or more, and it is difficult to apply the steering angle in stages.

### DISCLOSURE

### Technical Problem

It is an object of the present invention to provide a flexible catheter using a wire which solves the problems of conventional flexible catheters described above.

### Technical Solution

In accordance with an aspect of the present invention, provided is a flexible catheter including an insertion unit including a plurality of bending portions arranged in a longitudinal direction, an operation unit connected to the insertion unit, a direction adjustment wire having one end connected to the operation unit and a remaining end fixed to the insertion unit, the direction adjustment wire being configured to be capable of bending the bending portions according to a length pulled by the operation unit, and a limiter provided on the direction adjustment wire and configured to allow the respective bending portions to be sequentially bent as the direction adjustment wire is pulled.

Here, the bending portions may include a first bending portion and a second bending portion that are formed in sequence from a tip end of the insertion unit, and the first bending portion and the second bending portion may be configured such that the first bending portion is bent for a first period as the direction adjustment wire is pulled and the second bending portion is bent for a second period after the first period as the direction adjustment wire is pulled.

Then, each bending portion may be formed of a material having a lower strength than a remaining portion of the insertion unit to enable bending thereof.

Meanwhile, a holder may be fixedly provided between the first bending portion and the second bending portion, and the holder may support the limiter to prevent the limiter from passing through a position at which the holder is provided.

In addition, the holder may be formed at the position at which a distance between the holder and the limiter is reduced for the first period as the direction adjustment wire is pulled and at which the holder is kept in a state of being supported by the limiter for the second period.

Here, the holder may have a hollow shape and the direction adjustment wire may pass through the hollow-shaped holder.

Moreover, the holder may be configured as a protrusion that protrudes into a wire channel through which the direction adjustment wire is movable.

Here, at least a portion of the direction adjustment wire may be spirally disposed in a circumferential direction of the insertion unit so that torsion occurs in at least one bending portion among the plurality of bending portions.

In addition, the direction adjustment wire may be spirally disposed within the first bending portion and may be linearly disposed in the longitudinal direction from the holder to the operation unit.

Then, the insertion unit may include a working channel formed in a central portion of a cross section thereof so as to extend in the longitudinal direction, and the direction adjustment wire may be provided in plural around the working channel to realize bending in a plurality of directions.

### Advantageous Effects

A flexible catheter using a wire according to the present invention is capable of realizing smooth steering over 180 degrees or more as well as steering to an angle optimized for a particular tissue owing to sequential bending of a plurality of bending portions thereof, which results in efficient and accurate implementation of a surgical operation.

### DESCRIPTION OF DRAWINGS

FIG. 1 is a plan view of a flexible catheter according to a first embodiment of the present invention.

FIGS. 2A and 2B are cross-sectional views of the first embodiment.

FIGS. 3A to 3E are views illustrating the use state of the first embodiment.

FIGS. 4A, 4B and 4C illustrate modifications of the first embodiment.

FIGS. 5A and 5B are conceptual views of the operation of a second embodiment.

FIGS. 6A and 6B are cross-sectional views of a third embodiment.

FIG. 7 is a view illustrating the use state of the third embodiment.

### BEST MODE

Hereinafter, a flexible catheter using a wire according to embodiments of the present invention will be described in detail with reference to the accompanying drawings. In the following description of the embodiments, the names of respective components may be referred to as other names in the art. However, when there is functional similarity or identity of the components, the components may be regarded as being equivalent even if embodiments in which the components are modified are employed. In addition, reference numerals are added to the respective components for convenience of explanation. However, illustrations of the drawings in which these reference numerals are described do not limit the respective components to the range within the drawings. Likewise, even if embodiments in which the components illustrated in the drawings are partially modified are employed, the components may be regarded as being equivalent when there is functional similarity or identity of the components. Further, when a component is recognized as a component to be naturally included in view of the level of ordinary skills in the art, a description thereof will be omitted. In addition, in the drawings described below, some components may be exaggerated or omitted for convenience of explanation, and the technical idea thereof is not excluded from the scope of the right.

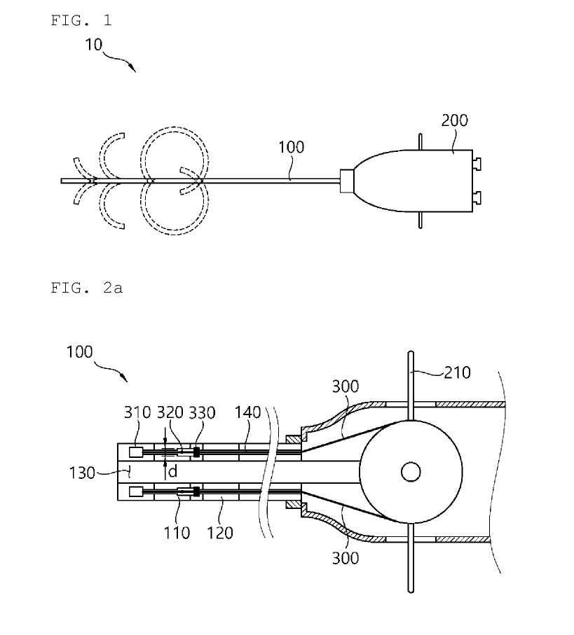

FIG. 1 is a plan view of flexible catheter 10 according to a first embodiment of the present invention.

As illustrated, the flexible catheter 10 according to the present invention may include an insertion unit 100, an operation unit 200, a direction adjustment wire 300, and a limiter 320.

The insertion unit 100 is configured to be able to be inserted into a tissue or a body cavity. The insertion unit 100 is elongated to extend in the longitudinal direction so as to be accessible to the body cavity or the tissue in a deep body region. A plurality of working channels 130 may be formed in the longitudinal direction within the insertion unit 100. Each working channel 130 may be connected to a port in the operation unit 200 to allow an optical fiber, which photographs the inside of the body cavity, or a treatment unit, which performs treatment on a lesion site, to be introduced from the operation unit 200, which will be described later, and protrude from a tip end of the insertion unit 100. In order to ensure smooth bending of the insertion unit 100, the insertion unit 100 may have a diameter equal to or less than 3 mm.

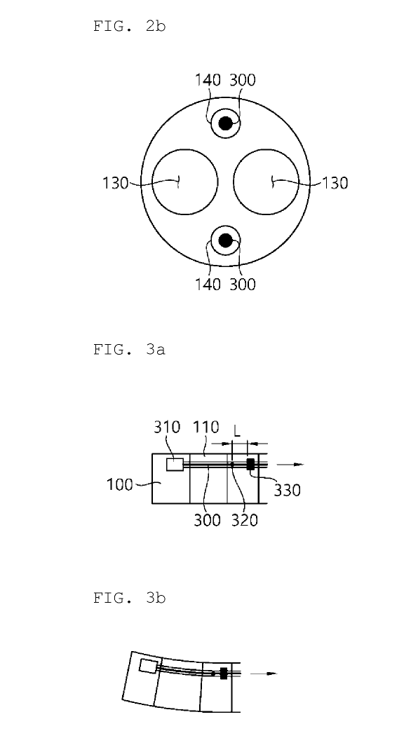

As illustrated in FIG. 1, the insertion unit 100 may include a plurality of bending portions, and as the direction adjustment wire 300 is pulled, the steering angle thereof may be determined and the bending portions may be sequentially bent. The operation of the wire and the steering angle will be described later in detail with reference to FIGS. 2 to 5.

The operation unit 200 may be connected to one end of the insertion unit 100, and an operator may perform surgery or diagnosis in the state of gripping the operation unit 200. The operation unit 200 may include a housing, a steering lever 210, and a connection port.

The housing defines the overall external appearance of the operation unit 200, and is shaped easily gripped by hand. The housing may have a somewhat curved shape in the longitudinal direction in order to prevent slip of the hand when the operator reciprocally moves the flexible catheter 10 forward and rearward into or out of the tissue in the state of gripping the flexible catheter 10.

The steering lever 210 is provided inside the housing. A portion of the lever protrudes outward from the housing to allow the operator to bend the insertion unit 100 by operating the steering lever 210 in the state of gripping the steering lever 210 by hand. The steering lever 210 is rotatably connected to the housing, and the direction adjustment wire 300 is mounted on one side of the steering lever 210 so that the steering angle of the insertion unit 100 is determined according to the amount of rotation of the steering lever 210. Here, the limit of the operation of the steering lever 210 may be set in various ways to correspond to the length of the bending portions of the insertion unit 100. A further detailed description related to the steering lever 210 will be omitted since the steering lever 210 may have any of various mechanical configurations including a cam structure.

As described above, the connection port 220 is connected to the working channel 130 so that the treatment unit or the optical fiber may be inserted into the working channel. In addition, the connection port 220 is configured to be connectable to an external device so as to supply a fluid to the working channel 130 or suck the fluid from the working channel 130. In this case, a fastening structure such as a screw thread may be formed on the exterior of the connection port 220 for fixing the external device.

Hereinafter, the structure of the insertion unit 100 of the flexible catheter 10 according to the present invention will be described in detail with reference to FIG. 2.

FIG. 2 is a cross-sectional view of the first embodiment. As illustrated, the insertion unit 100 may include a first bending portion 110, a second bending portion 120, the working channel 130, a wire channel 140, the direction adjustment wire 300, the limiter 320, and a holder 330.

The first bending portion 110 and the second bending portion 120 are formed in sequence from the tip end of the insertion unit 100 that is to be inserted into a tissue, and may be steered as the direction adjustment wire 300 is pulled. The first bending portion 110 and the second bending portion 120 may be formed of a material that has relatively low rigidity and is easily bendable compared to the remaining portion of the insertion unit 100. That is, the entire insertion unit 100 is flexible, but the flexibility of the first bending portion 110 and the second bending portion 120 is relatively high, so that the respective bending portions are intensively steered when the direction adjustment wire 300 is operated. Here, the first bending portion 110 is formed of a material having lower rigidity than that of the second bending portion 120, so that the first bending portion 110 and the second bending portion 120 may be easily sequentially bent.

The tip end of the insertion unit 100 is formed of a material having higher rigidity than that of the first bending portion 110 and the second bending portion 120, so that the insertion unit may be inserted without any significant deformation even if frictional contact shock is applied to the end thereof upon insertion.

The working channel 130 forms a path through which a treatment unit or an endoscope inserted from the outside moves, and is formed in the longitudinal direction within the entire insertion unit. The working channel 130 may be formed of any of various diameters according to the type of treatment. In the present embodiment, the case in which two working channels are formed will be described. Referring again to FIG. 2B, two working channels 130 are arranged in the horizontal direction and two wire channels 140 are arranged in the vertical direction, so that a plurality of channels may be formed in a compact space.

Each wire channel 140 may be configured such that the direction adjustment wire 300, which will be described later, may be disposed therein, and may have an inner diameter slightly greater than the diameter the direction adjustment wire 300. The wire channel 140 is formed to extend from the vicinity of the tip end of the insertion unit to the operation unit, and is open at one side thereof so as to be connected to the operation unit 200. The wire channel 140 is not formed in the tip end of the insertion unit, which may prevent contamination of the wire channel due to external impurities, for example. A portion of the wire channel 140 in which the limiter 320, which will be described later, reciprocates may have a somewhat great inner diameter d in order to ensure smooth movement of the limiter.

Two direction adjustment wires 300 are arranged to realize bending in opposite directions. The direction adjustment wires 300 are disposed in the respective wire channels 140 which are formed in the longitudinal direction near the working channels 130 as described above.

The limiter 320 is provided at one position on each direction adjustment wire 300 inside the first bending portion 110. The limiter 320 is configured to allow the first bending portion 110 and the second bending portion. 120 to be sequentially bent as the direction adjustment wire 300 is pulled. The holder 330 is provided between the first bending portion 110 and the second bending portion 120 to support the limiter 320. Although an example in which the holder 330 has a hollow shape so that the direction adjustment wire 300 may be inserted into and be moved through the holder is illustrated, this is merely given by way of example. The holder may be modified, for example, into a protrusion that protrudes into the wire channel 140 in order to prevent the limiter 320 from molding beyond a specific position.

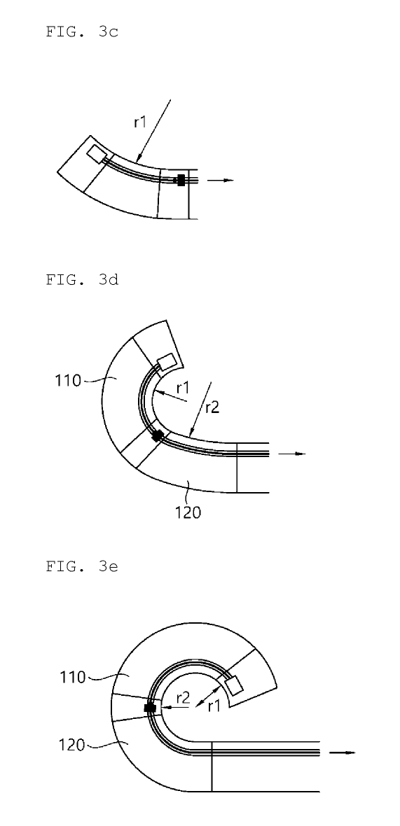

Hereinafter, the operation according to one embodiment of the present invention will be described in detail. FIG. 3 is a view illustrating the use state of the first embodiment. In FIG. 3, the sequence in which the respective bending portions are bent as the direction adjustment wire 300 is pulled is illustrated. The inner diameter of all of the wire channels 140 is illustrated as being greater than the diameter of the limiter 320 for convenience of explanation.

FIG. 3A illustrates an initial state in which the limiter 320 on the direction adjustment wire 300 and the holder 330 fixed to the insertion unit 100 are spaced apart from each other by a distance L. Referring to FIG. 3B, when the direction adjustment wire 300 starts to be pulled for a first period, the insertion unit starts to be bent upward since a fixing portion 310 is fixed thereto. At this time, the first bending portion 110 is a major portion taking charge of bending, and the limiter 320 moves toward the holder 330. Thereafter, when the direction adjustment wire 300 is further pulled, the limiter 320 is brought into contact with the holder 330 and the holder 330 supports the limiter 320 as illustrated in FIG. 3C. At this time, the fixing portion 310 is no longer p lied inside the first bending portion 110 and the first bending portion remains stationary at a specific curvature r1. The distance between the holder 330 and the limiter 320 may be determined to a distance by which the first bending portion 110 may be bent to 180 degrees. That is, as the distance between the holder 330 and the limiter 320 is increased, the bending angle of the first bending portion 110 becomes close to 180 degrees. As the distance between the holder 330 and the limiter 320 is reduced, the bending angle of the first bending portion 110 becomes smaller than 180 degrees.

As illustrated in FIG. 3D, when the direction adjustment wire 300 is further pulled for a second period, the limiter 320 supports the holder 330 and the second bending portion 120 starts to be bent. That is, from the moment when the limiter 320 is supported by the holder 330, the bending of the second bending portion 120 is performed in the state in which the first bending portion 110 is kept at a fixed degree of bending. At this time, the curvature r2 of the second bending portion 120 may vary according to the degree of pulling. As illustrated in FIG. 3E, the steering angle that enables the maximum bending of the first bending portion 110 may be about 180 degrees and the steering angle that enables the maximum bending of the second bending portion 120 may be about 180 degrees, whereby the overall steering angle may be close to 360 degrees. Although not illustrated, when the pulled direction adjustment wire 300 is released, motion in the reverse order of the above-described order may occur.

The above-described operating state is a description related to one direction adjustment wire 300, and steering in an opposite direction is possible. In addition, bending is possible in various directions when three or more direction adjustment wires 300 are provided.

In addition, a plurality of bending portions may be provided to enable bending at three or more positions. In this case, the holder 30 and the limiter 320 may be provided to correspond to the number of bending portions.

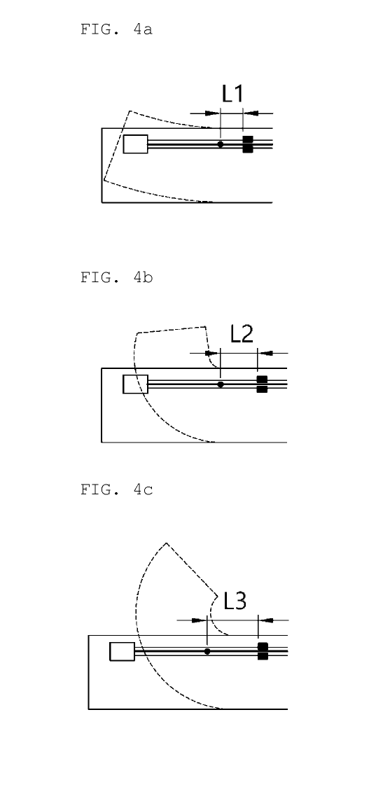

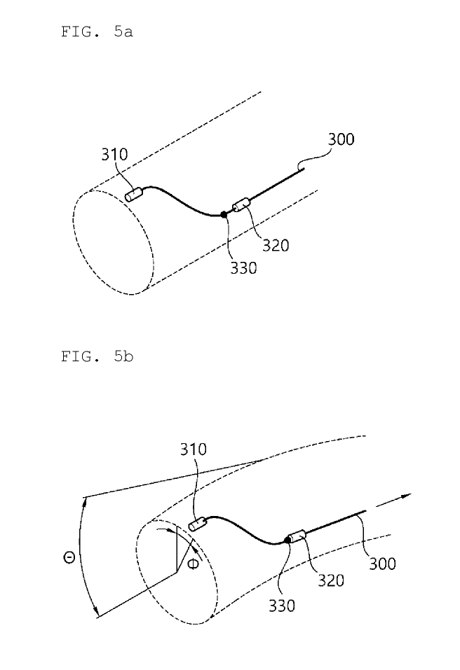

Hereinafter, modifications and other embodiments of the flexible catheter 10 according to the present invention will be described in detail with reference to FIGS. 4 and 5.

FIGS. 4A, 4B and 4C illustrate modifications of the first embodiment. As illustrated, the distance between the holder 330 and the limiter 320 may be differently set (L1<L2<L3). The distance between the holder 330 and the limiter 320 may determine the bending angle of the first bending portion 110. The maximum value of the bending of the first bending portion 110 when the limiter 320 and the holder 330 are in contact with and supported by each other as the direction adjustment wire 300 is pulled is indicated by a dotted line. In addition, as the distance is increased, the first period becomes longer and the first bending portion 110 may be bent to a greater angle. In this way, the maximum value of the bending angle of the first bending portion. 110 may be structurally limited, and the length and the bending angle of the second bending portion 120 may also be limited, which enables combinations of various bending angles. Thus, it is possible to realize optimized bending that is specialized for specific tissue.

FIGS. 5A and 5B are conceptual views illustrating the configuration and operation of a second embodiment.

As illustrated, a portion of the direction adjustment wire 300 may be configured to wind the insertion unit 100 in the circumferential direction of the insertion unit 100. The direction adjustment wire 300 is configured to cause bending as well as torsion in the state in which the catheter is inserted. Referring to FIG. 5A, a portion of the direction adjustment wire 300 located in the first bending portion 110 is spirally disposed, and is linearly disposed in the longitudinal direction at the downstream side of the holder 330. Referring to FIG. 53, as the direction adjustment wire 300 is pulled, the limiter 320 moves toward the holder 330. At this time, the first bending portion 110 is bent to a predetermined angle θ such that the distance between the fixing portion 310 and the holder 330 is reduced, and at the same time, undergoes torsion to a predetermined angle ϕ. Thus, the catheter may rotate while bending a treatment unit and may undergo torsion to various angles according to the angle at which the fixing portion 310 and the holder 330 are disposed.

Although the example in which torsion occurs in the first bending portion 110 has been described, the angle of the holder 330 provided in the second bending portion 120 or any other bending portion may be changed to cause torsion for a specific period.

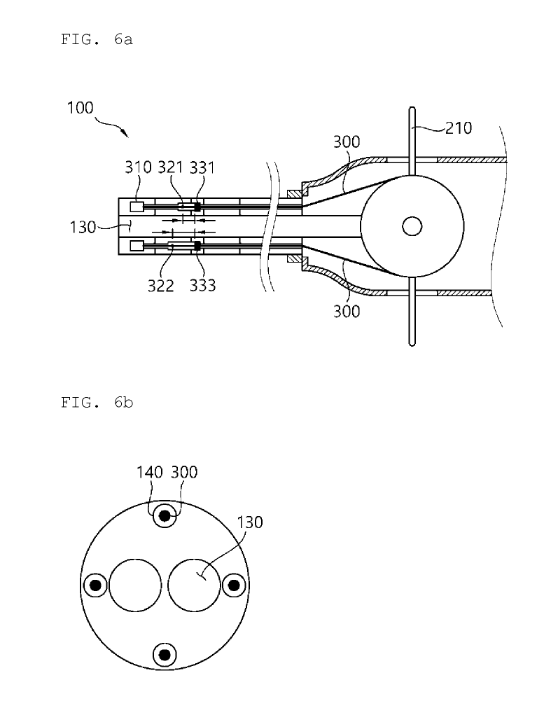

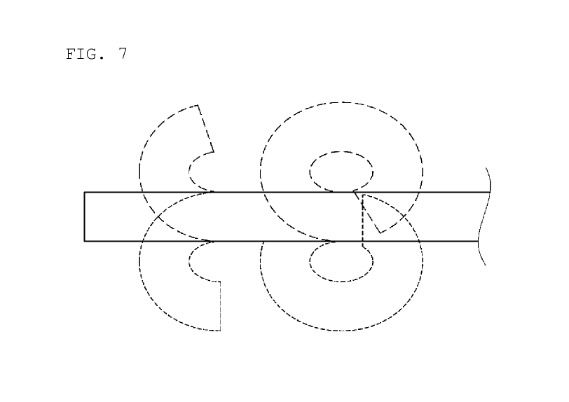

Hereinafter, a third embodiment will be described in detail with reference to FIGS. 6 and 7. In the present embodiment, the same elements as those in the other embodiments are included, and a description thereof will be omitted in order to avoid redundant description.

FIGS. 6A and 6B are cross-sectional views of a third embodiment. FIG. 7 is a view illustrating the use state of the third embodiment. As illustrated, direction adjustment wires are arranged at the interval of 90 degrees in upper, lower, left, and right directions so as to realize bending in four directions. Although not illustrated, in this case, in order to adjust the four direction adjustment wires, a plurality of operation units may be provided, or a joystick may be provided.

In the present embodiment, the position of a limiter on some of a plurality of direction adjustment wires may be different from the positions of the other limiters, so that the amount of bending of the first bending portion 110 may be adjusted differently. That is, the distance between a first holder 331 and a first limiter 321 is less than the distance between a second holder 332 and a second limiter 322. Thus, since the position at which the holder is supported by the limiter is different even when the same length is pulled, the steering angle in the downward direction in which the distance increased may be increased. Meanwhile, since the steering angle of the first bending portion 110 is fixed after the first holder 331 and the first limiter 321 or the second holder 332 and the second limiter 322 are supported by each other, the steering angle when the direction adjustment wire is pulled to the maximum extent is also changed, as illustrated in FIG. 7. Thus, the catheter may be rotated so as to be used at a specific steering angle when it is attempted to bend the catheter to a certain angle as needed.

As described above, in the flexible catheter 10 according to the present invention, the insertion unit 100 includes a plurality of bending portions and the limiter 320 and the holder 330 are provided to allow the plurality of bending portions to be sequentially bent, whereby bending may be realized at various angles and bending to 180 degrees or more may be implemented.

1. A flexible catheter comprising:
an insertion unit including a plurality of bending portions arranged in a longitudinal direction;
an operation unit connected to the insertion unit;
a direction adjustment wire having one end connected to the operation unit and a remaining end fixed to the insertion unit, the direction adjustment wire being configured to be capable of bending the bending portions depending on a length pulled by the operation unit; and
a limiter provided on the direction adjustment wire and configured to allow respective bending portions to be sequentially bent as the direction adjustment wire is pulled.

2. The catheter according to claim 1, wherein:
the bending portions include a first bending portion and a second bending portion that are formed in sequence from a tip end of the insertion unit; and
the first bending portion and the second bending portion are configured such that the first bending portion is bent for a first period as the direction adjustment wire is pulled, and the second bending portion is bent for a second period after the first period as the direction adjustment wire is pulled.

3. The catheter according to claim 2, wherein each bending portion is formed of a material having a lower strength than a remaining portion of the insertion unit to enable bending thereof.

4. The catheter according to claim 2, further comprising a holder fixedly provided between the first bending portion and the second bending portion,
wherein the holder and the limiter are supported by each other to prevent the limiter from passing through a position at which the holder is provided.

5. The catheter according to claim 4, wherein the holder is formed at the position at which a distance between the holder and the limiter is reduced for the first period as the direction adjustment wire is pulled and at which the holder is kept in a state of being supported by the limiter for the second period.

6. The catheter according to claim 5, wherein the holder and the limiter are spaced apart from each other by the distance by which the first bending portion is bendable to 180 degrees.

7. The catheter according to claim 4, wherein the holder has a hollow shape and the direction adjustment wire passes through the hollow-shaped holder.

8. The catheter according to claim 4, wherein the holder is configured as a protrusion that protrudes into a wire channel through which the direction adjustment wire is movable.

9. The catheter according to claim 4, wherein at least a portion of the direction adjustment wire is spirally disposed in a circumferential direction of the insertion unit so that torsion occurs in at least one bending portion among the plurality of bending portions.

10. The catheter according to claim 9, wherein the direction adjustment wire is spirally disposed within the first bending portion and is linearly disposed in the longitudinal direction from the holder operation unit.

11. The catheter according to claim 2, wherein:
the insertion unit includes a working channel formed in a central portion of a cross section thereof so as to extend in the longitudinal direction; and
the direction adjustment wire is provided in plural around the working channel to realize bending in a plurality of directions.
